(19) United States
(12) Patent Application Publication
(10) Pub. No.: US 2018/0168484 A1
(43) Pub. Date: Jun. 21, 2018

(54) PULMONARY FUNCTION TEST DEVICES AND METHODS

(71) Applicant: N E FIELD DIAGNOSTICS LTD., Kfar Saba (IL)

(72) Inventors: Ofer RAHAMIM, Kfar Saba (IL); Vladimir KOTLER, Kfar Saba (IL); David SHOSEYOV, Karmei Yosef (IL); Benita LANZER, Karkur (IL); Yoav ROSENBACH, Ramat Gan (IL)

(21) Appl. No.: 15/574,215

(22) PCT Filed: May 17, 2016
(86) PCT No.: PCT/IL2016/050521

§ 371 Date: Nov. 15, 2017

Related U.S. Application Data
(60) Provisional application No. 62/162,747, filed on May 17, 2015.

(51) Int. Cl. A61B 5/097; A61B 5/087; A61B 5/00; A61B 5/085; A61M 16/16; A61B 5/08

(52) U.S. Cl. CPC A61B 5/097; A61B 5/0878; A61B 5/7278; A61B 5/085; A61B 2560/0257; A61M 16/16; A61B 5/0803; A61B 2560/0252; A61M 2205/3372; A61B 5/4818

(57) ABSTRACT

The present disclosure introduces a pulmonary function testing device, the device includes at least one sensor for enabling airflow measurements of gas flow within the device, a respiratory characteristic modulator configured to change between at least a first respiratory characteristic and a second respiratory characteristic and a processing circuitry for derive the respiratory related parameter based on measurements of flow obtained in at least a first and a second respiratory cycles utilizing at each of the first respiratory characteristic and a second respiratory characteristic respectively.

### TECHNICAL FIELD

The present disclosure generally relates to the field of pulmonary function test devices and methods.

### BACKGROUND

Pulmonary function tests (PFT's) are diagnostic tests that provide measurable data to evaluate the function of the lungs by assessing lung volumes, capacities, flow rates, and gas exchange. PFT's are performed during exhalation and inhalation, and are essential in diagnosis of certain lung disorders, such as asthma, bronchitis, chronic obstructive pulmonary disease, and other respiratory medical conditions in patients ranging from infants to the elderly.

Most of the current PFT devices require a high degree of cooperation from the patients, who cannot always comply; patients such as infants and/or unconscious patients and/or partially-conscious patients and/or comatose patients and/or patients under sedation or other medication. Such patients cannot understand and/or appropriately fulfill instructions such as “exhale forcefully” into a tube. Some patients that suffer acute pain or acute medical conditions cannot practically exhale and/or inhale forcefully through a tube or apparatus at full force and/or at the instructed breathing rate, which are common requirements when performing PFT's. Moreover, some PFT's performed on non-collaborative patients are lengthy, cumbersome and may require special devices such as a plethysmograph or other. Such devices are complex to operate, expensive, and require a high level of technical expertise for proper operation.

There is thus a need in the art for pulmonary function test devices that are easy to operate and require little to no degree of cooperation from patients.

### SUMMARY

The following embodiments and aspects thereof are described and illustrated in conjunction with systems, apparatus, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided herein devices, systems and methods for pulmonary function tests, including a controllable air-resistance structure having at least two selectable distinct air resistance characteristics (for example, air resistance values) and at least one airflow sensor for measuring the flow of air passing through the air resistance structure. The flow of air is measured while a subject breathes through two or more air flow resistors or at two or more different resistance characteristics, and a computational analysis is done in order to obtain the subjects' respiratory related parameters. Advantageously, the test may be conducted without any cooperation from the subject.

More technical terminology and definitions related to respiratory testing, parameters and measurements can be found at chapters 5 and 9, titled “MEASUREMENT OF FLOW AND VOLUME” and “PLETHYSMOGRAPHIC ASSESSMENT OF FUNCTIONAL RESIDUAL CAPACITY AND AIRWAY RESISTANCE” respectively, in publication/book “INFANT RESPIRATORY FUNCTION TESTING” by editors Janet Stocks, Peter D. Sly, Robert S. Tepper, and Wayne J. Morgan, Published by Wiley-Liss, 1996, which is hereby incorporated by reference. (ISBN 10: 0471076821/ISBN 13: 9780471076827).

More information about mechanics of breathing can be found at chapter 7, titled “MECHANICS OF BREATHING”, in publication/book “RESPIRATORY PHYSIOLOGY THE ESSENTIALS” by author John Burnard West, which is hereby incorporated by reference. (ISBN 10: 1609136403/ISBN 13: 9781609136406).

According to some embodiments, the resistance characteristics of the device to air flow should be low relative to the airway resistance of the subject, although values equal to or higher than the subjects' airway resistance may be used in order to conduct a test but may yield poorer result precision.

According to some embodiments, the device is automatic and the proper operation thereof requires only basic technical skills. Advantageously, the simplicity of the operation may enable a wider, more extensive use of pulmonary function testing, such as for screening tests, to provide early detection and treatment of respiratory conditions.

According to some embodiments, the device may be configured to monitor continuous breathing and provide results and indications of the subjects' pulmonary functions. According to some embodiments, the device, system and method disclosed herein may be used, for example, in sleep laboratories for breath monitoring in order to diagnose, for example, sleep disorders such as sleep apnea, obstructive sleep apnea and other diseases or conditions.

According to some embodiments, there is provided a pulmonary function testing device for evaluating a respiratory related parameter of a subject, the device includes a controllable air-resistance structure having at least first and second selectable distinct air resistance characteristics, an actuator configured to control the air resistance structure and select the desired air resistance characteristics from the at least first and second distinct air resistance characteristics, at least one airflow sensor configured to provide a signal indicative of the flow of air passing through the air resistance structure, and processing circuitry.

According to some embodiments, the processing circuitry is configured to assign the first air resistance characteristics for a first respiration cycle and obtain a first signal indicative of the flow of air passing through the air resistance structure having the first air resistance characteristics during the first respiration cycle, assign the second air resistance characteristics for a second respiration cycle and obtain a second signal indicative of the flow of air passing through the air resistance structure having the second air resistance characteristics during the second respiration cycle, and derive the respiratory related parameter based at least on the first and the second signals at corresponding respiratory phases.

According to some embodiments, respiratory phases are intervals within a respiratory cycle.

According to some embodiments, corresponding respiratory phases are defined by breath volume.

According to some embodiments, the respiratory related parameter includes an airway resistance.

According to some embodiments, the respiratory related parameter includes changes in lung pressure corresponding to a respiratory cycle.

According to some embodiments, the respiratory related parameter includes changes in lung compliance corresponding to a respiratory cycle.

According to some embodiments, the resistance characteristics include one or more air resistance values.

According to some embodiments, the resistance characteristics include flow dependent resistance values.

According to some embodiments, the device further includes a first temperature sensor configured to measure a temperature of air inhaled or exhaled by the subject, for example, in the device.

According to some embodiments, the device further includes a second temperature sensor configured to measure a temperature of an environment surrounding the device.

According to some embodiments, the device further includes a temperature controller configured to control a temperature in the device.

According to some embodiments, the device further includes an ambient pressure sensor configured to measure pressure of a surrounding environment of the device.

According to some embodiments, the device further includes a differential pressure sensor configured to measure a pressure difference between two points, one of which is located in a mouthpiece or between the mouthpiece and the modulator.

According to some embodiments, the device further includes a differential pressure sensor configured to measure pressure in the device.

According to some embodiments, the processing circuitry is further configured to generate one or more graphs depicting, a representative respiratory cycle, airway resistance, alveolar pressure, lung compliance or any combination thereof.

There is provided herein, according to some embodiments, a method for pulmonary function testing for evaluating respiratory related parameter of a subject, the method including providing a device including a controllable air-resistance structure having at least first and second selectable distinct air resistance characteristics, assigning the first air resistance characteristics for a first respiration cycle and obtaining a first signal indicative of the flow of air passing through the air resistance structure having the first air resistance characteristics during the first respiration cycle, measuring air flow through the device during the first respiration, assigning the second air resistance characteristics for a second respiration cycle and obtaining a second signal indicative of the flow of air passing through the air resistance structure having the second air resistance characteristics during the second respiration cycle, measuring air flow through the device during the second respiration, and deriving, using processing circuitry, the respiratory related parameter based at least on the first and the second signals at corresponding respiratory phases.

According to some embodiments, respiratory phases are intervals within a respiratory cycle.

According to some embodiments, corresponding respiratory phases are defined by breath volume.

According to some embodiments, the respiratory related parameter includes an airway resistance.

According to some embodiments, the respiratory related parameter includes changes in lung pressure corresponding to a respiratory cycle.

According to some embodiments, the respiratory related parameter includes changes in lung compliance corresponding to a respiratory cycle.

According to some embodiments, the resistance characteristics include one or more air resistance characteristics.

According to some embodiments, the resistance characteristics include flow dependent resistance characteristics.

According to some embodiments, the method further includes measuring a temperature of air in the device.

According to some embodiments, the method further includes measuring a temperature of an environment surrounding the device.

According to some embodiments, the method further includes controlling a temperature in the device.

According to some embodiments, the method further includes measuring ambient pressure of a surrounding environment of the device.

According to some embodiments, the method further includes measuring a pressure difference between two points, one of which is within the device or is located in a mouthpiece or between the mouthpiece and the modulator.

According to some embodiments, the method further includes measuring pressure in the device.

According to some embodiments, the method further includes using processing circuitry, generating one or more graphs depicting, a representative respiratory cycle, airway resistance, alveolar pressure, lung compliance or any combination thereof.

There is also provided herein, according to some embodiments, a pulmonary function testing system for evaluating a respiratory related parameter of a subject, the device including a controllable air-resistance structure having at least first and second selectable distinct air resistance characteristics, an actuator configured to control the air resistance structure and select the desired air resistance characteristics from at least first and second distinct air resistance characteristics, at least one airflow sensor configured to provide a signal indicative of the flow of air passing through the air resistance structure, and processing circuitry.

The processing circuitry is configured to assign the first air resistance characteristics for a first respiration cycle and obtain a first signal indicative of the flow of air passing through the air resistance structure having the first air resistance characteristics during the first respiration cycle, assign the second air resistance characteristics for a second respiration cycle and obtain a second signal indicative of the flow of air passing through the air resistance structure having the second air resistance characteristics during the second respiration cycle, and derive the respiratory related parameter based at least on the first and the second signals at corresponding respiratory phases;

According to some embodiments, the system further includes a monitor configured to display test related data.

According to some embodiments, the test related data include the respiratory related parameter, at least first and second distinct air resistance characteristics, patient information, operation instruction or any combination thereof.

As used herein, according to some embodiments, the term “respiratory” may be interchangeable with the terms pulmonary, lung or lung-related, airways or airways-related.

According to some embodiments, the system further includes a user interface breath unit, such as a nozzle, a mask, a mouthpiece, or other structures configured to facilitate obtaining and providing respiratory gas from and to the user, respectively, through the device.

According to some embodiments, the user interface breath unit may include, a nozzle, a mask, a mouthpiece, a tube or other structures configured to facilitate obtaining and providing respiratory gas from and to the user, respectively, through the device.

According to some embodiments, there is provided herein a pulmonary function testing device for evaluating a respiratory related parameter of a subject, the device comprising: at least one sensor configured to provide a signal, directly or indirectly, indicative of the flow rate of air passing through the device, a respiratory characteristic modulator configured to change between at least a first respiratory characteristic and a second respiratory characteristic, wherein the respiratory characteristics are selected from a group consisting of: compliance, flow and pressure; and processing unit configured to: obtain from the sensor a first signal acquired during respiration, while the first respiratory characteristic is set; obtain from the sensor a second signal acquired during respiration while the second respiratory characteristic is set; and derive the respiratory related parameter based at least on the first and the second signals.

According to some embodiments, there is provided a pulmonary function testing device, the device includes at least one sensor configured to provide a signal, directly or indirectly, indicative of the flow rate of air passing through the device, a respiratory characteristic modulator configured to change between at least a first respiratory characteristic and a second respiratory characteristic, and a processing unit configured to, obtain from the sensor a first signal acquired during a first respiration cycle or part thereof and while the first respiratory characteristic is set, obtain from the sensor a second signal acquired during the second respiration cycle or part thereof and while the second respiratory characteristic is set, and derive the respiratory related parameter based at least on the first and the second signals at corresponding respiratory cycles or parts thereof.

According to some embodiments, the respiratory characteristics are selected from a group consisting of: resistance, compliance, flow and pressure. According to some embodiments, the respiratory characteristics include resistance, wherein the first respiratory characteristics include a first resistance, and the second respiratory characteristics include a second resistance. According to some embodiments, the respiratory characteristics are selected from a group consisting of: compliance, flow and pressure, and the first respiratory characteristics and the second respiratory characteristics include one or more of compliance, flow and pressure. According to some embodiments, the respiratory characteristic modulator is an air resistance structure configured to apply a first air resistance characteristic to air passes therethrough during the first respiration cycle or part thereof and a second air resistance characteristic to air passes therethrough during the second respiration cycle or part thereof.

According to some embodiments, the at least one sensor includes a pneumotach.

According to some embodiments, there is provided herein, a pulmonary function testing device, the device comprising: at least one sensor configured to provide a signal, directly or indirectly, indicative of the flow rate of air passing through said device, a resistance modulator configured to change between at least a first resistance and a second resistance; and a processing unit configured to: obtain from said sensor a first signal acquired during a first respiration cycle or part thereof and while the first resistance is set; obtain from said sensor a second signal acquired during said second respiration cycle or part thereof and while the second resistance is set, and derive the respiratory related parameter based at least on the first and the second signals at corresponding respiratory cycles or parts thereof, wherein the respiratory related parameter may include airway resistance of the subject's internal airway path, lung pressure, changes in lung pressure, lung compliance, changes in lung compliance, alveolar pressure, changes in alveolar pressure or any combination thereof. Each possibility presents a separate embodiment.

According to some embodiments, the device further includes an actuator configured to control the respiratory characteristic modulator and to apply at least the first and the second respiratory characteristics. According to some embodiments, the respiratory related parameter includes an airway resistance of the subject's internal airway path. According to some embodiments, the respiratory related parameter includes lung pressure. According to some embodiments, the respiratory related parameter includes changes in lung pressure. According to some embodiments, the respiratory related parameter includes lung compliance. According to some embodiments, the respiratory related parameter includes changes in lung compliance. According to some embodiments, the respiratory related parameter includes alveolar pressure.

According to some embodiments, the respiratory related parameter includes changes in alveolar pressure. According to some embodiments, the device further includes a temperature sensor configured to measure a temperature of air inhaled or exhaled by the subject, for example, but not limited to, in the device. According to some embodiments, the device further includes a temperature sensor configured to measure a temperature of an environment surrounding the device. According to some embodiments, the device further includes a temperature module configured to control a temperature of air inhaled by the subject. According to some embodiments, the temperature module is configured to heat the air inhaled by the subject. According to some embodiments, the device further includes a humidity module configured to control humidity of air inhaled by the subject. According to some embodiments, the device further includes a pressure sensor configured to measure pressure of a surrounding environment of the device. According to some embodiments, the device further includes one or more pressure sensors configured to measure a pressure difference between two points, one of which is located in a mouthpiece or between the mouthpiece and the modulator.

According to some embodiments, the device further includes two or more pressure sensors configured to measure a pressure difference between two points, one of which is within the device or located in a mouthpiece or between the mouthpiece and the modulator. According to some embodiments, the processing unit is further configured to generate one or more displays/graphs depicting a representative respiratory cycle, airway resistance, alveolar pressure, lung compliance or any combination thereof. According to some embodiments, the device further includes a display unit. According to some embodiments, the device further includes a user interface breath unit. According to some embodiments, the user interface breath unit includes a mask, a tube or both.

According to some embodiments, there is provided a pulmonary function testing system for evaluating a respiratory related parameter of a subject, the system includes a respiratory characteristic modulator configured to change between at least a first respiratory characteristic and a second respiratory characteristic, a pulmonary function testing device including at least one sensor configured to provide a signal, directly or indirectly, indicative of the flow rate of air passing through the device, and a processing unit configured to obtain from the sensor a first signal acquired during a first respiration cycle or part thereof and while the first respiratory characteristic is set, obtain from the sensor a second signal acquired during the second respiration cycle or part thereof and while the second respiratory characteristic is set, and derive the respiratory related parameter based at least on the first and the second signals at corresponding respiratory cycles or parts thereof.

According to some embodiments, the at least one sensor includes a pneumotach. According to some embodiments, the respiratory characteristics are selected from a group consisting of: resistance, compliance, flow and pressure. According to some embodiments, the respiratory characteristics include resistance, wherein the first respiratory characteristics include a first resistance, and the second respiratory characteristics include a second resistance. According to some embodiments, the respiratory characteristics are selected from a group consisting of: compliance, flow and pressure.

According to some embodiments, the respiratory characteristic modulator is an air resistance structure configured to apply a first air resistance characteristic to air passing therethrough during the first respiration cycle or part thereof and a second air resistance characteristic to air passing therethrough during the second respiration cycle or part thereof. According to some embodiments, the system further includes an actuator configured to control the respiratory characteristic modulator and to apply at least the first and the second respiratory characteristics. According to some embodiments, the respiratory related parameter includes an airway resistance of the subject's internal airway path. According to some embodiments, the respiratory related parameter includes lung pressure. According to some embodiments, the respiratory related parameter includes changes in lung pressure.

According to some embodiments, the respiratory related parameter includes lung compliance. According to some embodiments, the respiratory related parameter includes changes in lung compliance. According to some embodiments, the respiratory related parameter includes alveolar pressure. According to some embodiments, the respiratory related parameter includes changes in alveolar pressure. According to some embodiments, the system further includes a temperature sensor configured to measure a temperature of air in the device. According to some embodiments, the system further includes a temperature sensor configured to measure a temperature of an environment surrounding the device.

According to some embodiments, the system further includes a temperature module configured to control a temperature of air inhaled by the subject. According to some embodiments, the temperature module is configured to heat the air inhaled by the subject. According to some embodiments, the system further includes a humidity module configured to control humidity of air inhaled by the subject. According to some embodiments, the system further includes a pressure sensor configured to measure pressure of a surrounding environment of the device. According to some embodiments, the system further includes one or more pressure sensors configured to measure a pressure difference between two points, one of which is located in a mouthpiece or between the mouthpiece and the modulator. According to some embodiments, the system further includes two or more pressure sensors configured to measure a pressure difference between two points, one of which is within the device or is located in a mouthpiece or between the mouthpiece and the modulator.

According to some embodiments, the processing circuitry is further configured to generate one or more displays/graphs depicting a representative respiratory cycle, airway resistance, alveolar pressure, lung compliance or any combination thereof. According to some embodiments, the system further includes a display unit. According to some embodiments, the system further includes a user interface breath unit. According to some embodiments, the user interface breath unit includes a mask, a tube or both.

According to some embodiments, there is provided a method for pulmonary function testing for evaluating respiratory related parameter of a subject, the method includes providing a pulmonary function testing device including at least one sensor configured to provide a signal, directly or indirectly, indicative of the flow rate of air passing through the subject's airway, applying a first respiratory characteristic and obtaining from the sensor a first signal, directly or indirectly, indicative of the flow rate of air passing through the device during a first respiration cycle or part thereof, applying a second respiratory characteristic and obtaining a second signal, directly or indirectly, indicative of the flow rate of air passing through the device during the second respiration cycle or part thereof, and deriving the respiratory related parameter based at least on the first and the second signals at corresponding respiratory cycles or parts thereof.

According to some embodiments, the respiratory related parameter includes an airway resistance. According to some embodiments, the respiratory characteristics includes resistance, wherein the first respiratory characteristics includes a first resistance, and the second respiratory characteristics includes a second resistance. According to some embodiments, the respiratory related parameter is selected from the group consisting of an airway compliance, flow and pressure. According to some embodiments, the respiratory related parameter includes lung pressure. According to some embodiments, the respiratory related parameter includes changes in lung pressure. According to some embodiments, the respiratory related parameter includes lung compliance. According to some embodiments, the respiratory related parameter includes changes in lung compliance. According to some embodiments, the resistance characteristics include flow dependent resistance values. According to some embodiments, the method further includes measuring a temperature of air inhaled by the subject.

According to some embodiments, the method further includes measuring a temperature of an environment surrounding the device. According to some embodiments, the method further includes controlling a temperature of air inhaled by the subject. According to some embodiments, the method further includes heating the air inhaled by the subject. According to some embodiments, the method further includes controlling humidity of air inhaled by the subject. According to some embodiments, the method further includes measuring ambient pressure of a surrounding environment of the device. According to some embodiments, the method further includes measuring a pressure difference between two points, at least one of which is within the device or is located in a mouthpiece or between the mouthpiece and the modulator.

According to some embodiments, the method further includes measuring mouth and/or nose pressure. This may be accomplished by measuring pressure in or in proximity to a mouthpiece, or in the device between a mouthpiece and a resistor (such as a pneumotach). According to some embodiments, the method further includes using processing circuitry, generating one or more displays/graphs depicting, a representative respiratory cycle, airway resistance, alveolar pressure, lung compliance or any combination thereof. According to some embodiments, the method further includes displaying graphs depicting a representative respiratory cycle, airway resistance, alveolar pressure, lung compliance or any combination thereof.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown to scale. The figures are listed below.

FIG. 1 schematically illustrates a pulmonary function testing system, according to some embodiments;

FIG. 2a schematically illustrates a block diagram of a pulmonary function testing system having a variable air resistance structure and a flow meter with a mask, according to some embodiments;

FIG. 2b schematically illustrates a block diagram of a pulmonary function testing system having a variable air resistance structure and a flow meter with a mouthpiece, according to some embodiments;

FIG. 3a schematically illustrates a pulmonary function testing device having a variable air resistance structure, including a shutter, according to some embodiments;

FIG. 3b schematically illustrates an air resistance shutter of FIG. 3a at a first position, according to some embodiments;

FIG. 3c schematically illustrates an air resistance shutter of FIG. 3a at second position, according to some embodiments;

FIG. 4 schematically illustrates a pulmonary function testing device including multiple discs, according to some embodiments;

FIG. 5a schematically illustrates a pulmonary function testing device with a flow valve at a first position, according to some embodiments;

FIG. 5b schematically illustrates a pulmonary function testing device with a flow valve at a second position, according to some embodiments, and

FIG. 6 illustrates a flow chart of a method for testing pulmonary function using a variable air resistance structure, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Current pulmonary function devices (such as spirometry devices), systems and methods require a high degree of cooperation from the patients; (for example, the patient might be required to inhale and/or exhale at a specific pace and/or force) such degree of cooperation is not always obtainable with many patients such as infants, elderly patients, unconscious patients, semiconscious patients, patients in a comatose state, tranquilized/anesthetized patients, and/or people suffering from various respiratory conditions. The aforementioned groups of people may also need more frequent spirometry tests than other groups, or the rest of the population. This issue is also apparent when spirometry tests need to be conducted on animals (for example, in research), where cooperation is not to be expected.

Another issue with current spirometry devices, systems and methods is the high degree of patient effort and resulting fatigue or exhaustion from performing many forced respirations, rendering the application of spirometry inconvenient and in some cases even infeasible.

Additionally, current pulmonary function equipment is expensive and complex, and requires a relatively high degree of technical expertise for proper operation. As a result, the use of spirometry is limited to specific cases, and the potential benefits of widely available pulmonary function tests are not fully seized upon.

According to some embodiments, there is provided a pulmonary function device with a controllable air-resistance structure having at least two distinct air resistance characteristics, one of which may be the natural resistance of the device parts in which air flows and at least one airflow sensor for measuring the flow of all (or essentially all) the inhaled and exhaled air of the breathing subject as it is passing through the device. The flow of air is measured while a subject breathes through the device with at least two different resistance characteristics, and a computational analysis is done based on measurements associated with the two resistance characteristics for deriving at least one desired respiratory related parameter(s). Multiple respiratory related parameters including specific values, rates and/or comparisons may also be obtained. Advantageously, the test may be conducted with no need for any special cooperation (including from intentional cooperation that may be insufficient, through to no cooperation at all) from the subject, during regular undisrupted respirations of the subject.

According to some embodiments, the device selects a first air resistance characteristic, then it measures the flow rate of air through the air resistance structure across at least one respiration cycle, or a substantial portion of a respiration cycle, then the device selects a second air characteristic and measures the flow of air through the structure across at least one respiration cycle, or a substantial portion of a respiration cycle, and then the device conducts a computational analysis based on the measurements to derive desired respiratory parameters.

According to some embodiments, the characteristic of a flow resistor is in direct correlation with the fluid flow rate, therefore the resistance/characteristic may be changed either by physically altering the resistor structure or by changing the rate of fluid (breath gases) flow.

According to some embodiments, a third and/or further air resistance characteristic (s) may be used, and computational analysis may be performed, based on calculating measurements of a first and a third air resistance characteristic (s).

According to some embodiments, the term “resistance” and “resistor” may interchangeably be used with the terms “air resistance”/“air flow resistance” and “air resistor”/“air flow resistor” as well as “air restrictor”/“air flow restrictor” and “air restrictor”/“air flow restrictor”. According to some embodiments, the term “characteristics” may include resistance, pressure, compliance, flow, air obstruction, partial obstruction and restriction and other characteristics that affect/determine a gas-flow behavior.

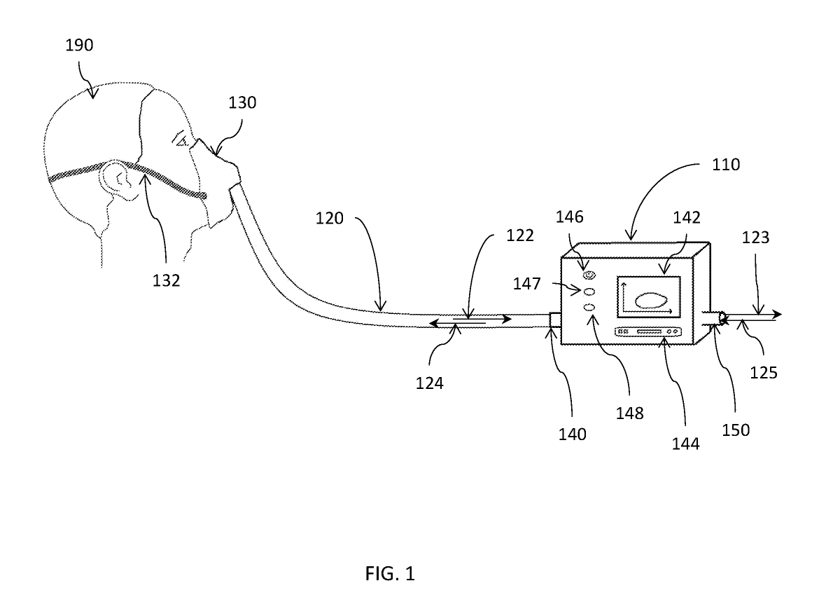

Reference is now made to FIG. 1, which schematically illustrates a pulmonary function testing system, according to some embodiments. The system includes a pulmonary function testing device 110, a tube 120 and a subject interface, shown as mask 130 configured for obtaining respiratory gas from a user 190 and to be mounted thereon using a strap 132. Tube 120 is connected at one end thereof to mask 130 and on the other end thereof to pulmonary function testing device 110. Pulmonary function testing device 110 has an inlet 140 configured to receive tube 120 and an outlet 150. The subject's exhaled breath is collected by mask 130 and is transferred through tube 120 (arrow 122) and through inlet 140 to pulmonary function testing device 110. A pulmonary function test is performed by pulmonary function testing device 110 and the exhaled breath then exits the device through outlet 150 (arrow 123). It is noted when the subject inhales, ambient air is sucked into pulmonary function testing device 110 through outlet 150 (arrow 125) and continues to the subject's respiratory opening, for example mouth and/or nostril(s), through tube 120 (arrow 124), through inlet 140 and through mask 130. Pulmonary function testing device 110 typically includes a display and/or operation panel.

According to some embodiments, the structure referred to as inlet 140 comprises an aperture/opening/orifice configured to provide respiratory gas from a user to the device during exhale, and from the device to the user during inhale. According to some embodiments, the structure referred to as outlet 150 comprises an aperture/opening/orifice configured to exhaust respiratory gas from a device to the surroundings thereof (ambient air) during exhale, and to provide gas from the surrounding of the device (ambient air) to the device during inhale.

According to some embodiments, function testing device 110 may include user-interface control and/or display(s), such as On/Off button 146, monitor 142 for providing graphical depiction of respiratory related information, a menu button 147, toggle button 144, for example, for controlling monitor 142, and light indicator(s) 148.

According to some embodiments, the term respiratory opening may refer to any opening/aperture that provides fluid respiratory gas flow to and from the lungs of the user, which may be accessed by an external user interface breath unit. The respiratory opening may be a mouth, nostril(s), or a surgical respiratory opening, and all occurrences of any of the terms mouth and or nostril(s), are interchangeable with the term respiratory opening.

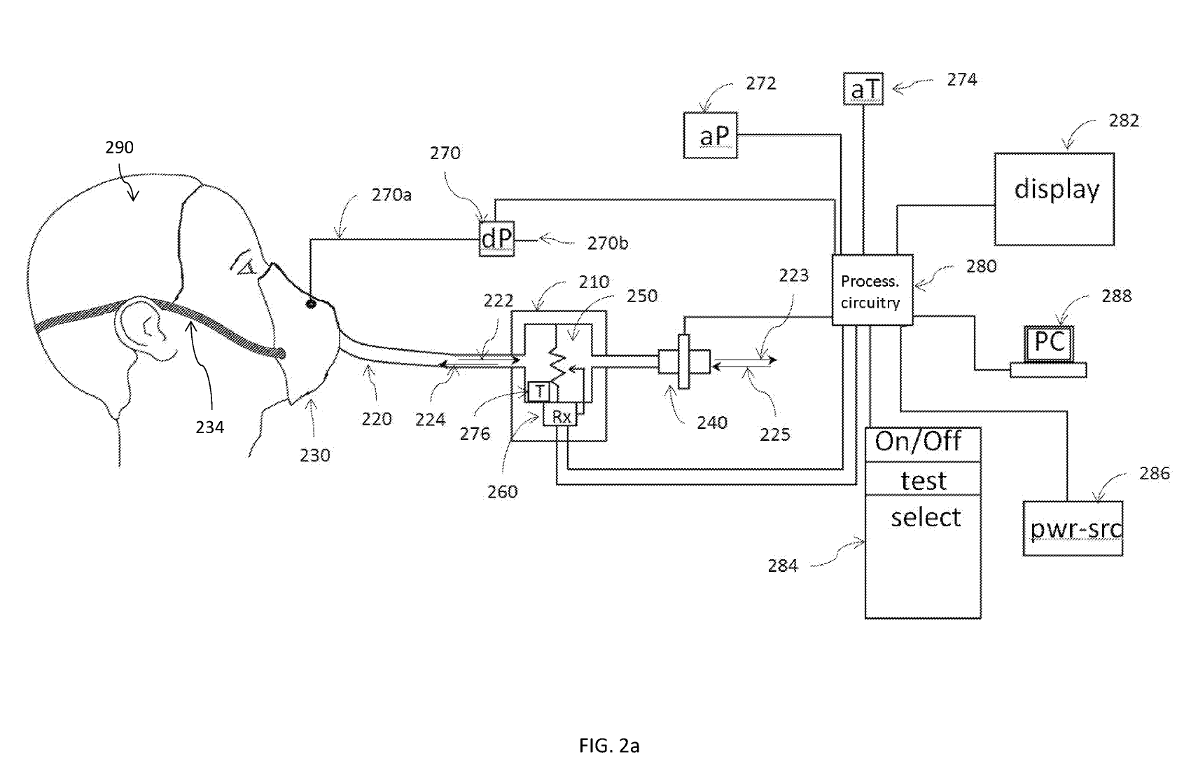

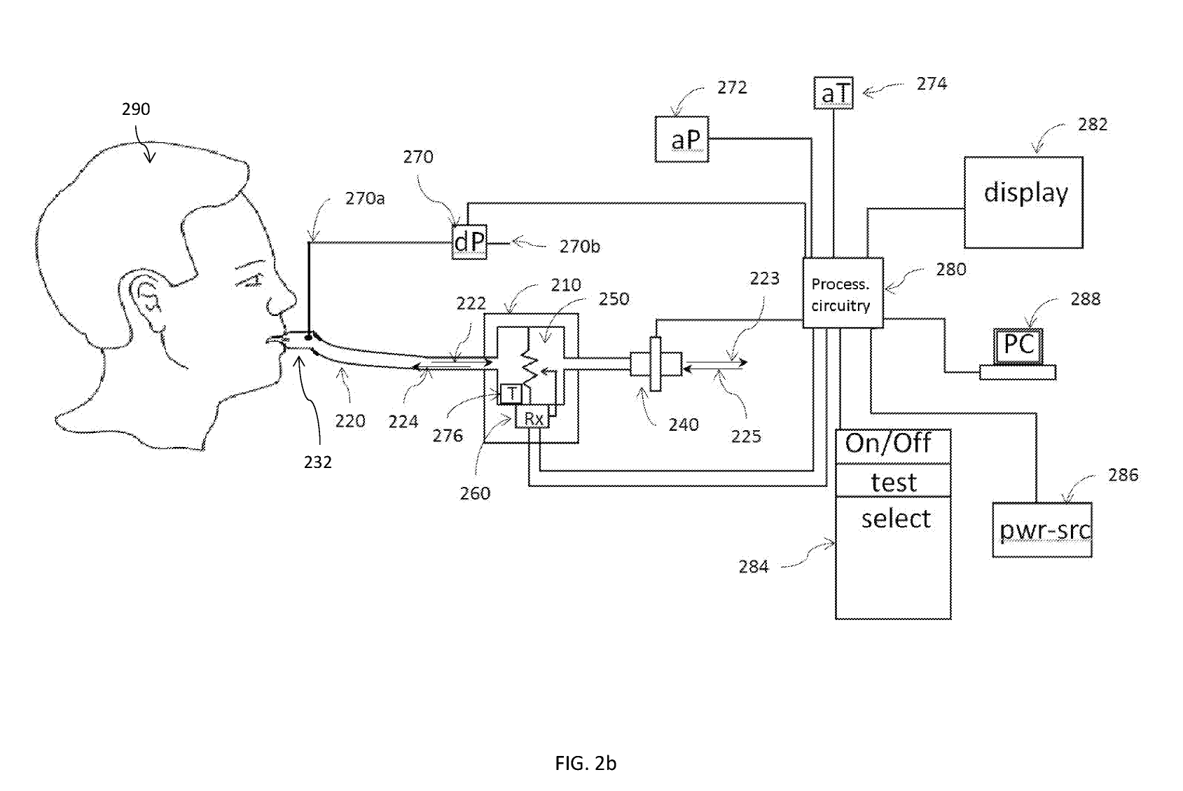

Reference is now made to FIG. 2a, which schematically illustrates a block diagram of a pulmonary function testing system having a mask 230 for providing respiratory gas from a user 290, a variable air resistance structure 210 and a flow meter 240, according to some embodiments. Variable air resistance structure 210 is in fluid flow (breath) communication with a subject interface, shown herein as a mask 230 and a strap 234 for fastening/mounting mask 230 to user 290, and with flow meter 240. Variable air resistance structure 210 comprises an air flow resistor 250 and a resistance characteristic variation component 260 which are configured to provide variable (for example 2 or more, such as 3, 4, 5, or more) air resistance characteristics.

According to some embodiments, a variable air resistance structure may be a specific structure(s) in a pulmonary function testing device (shown herein as variable air resistance structure 210) or it may include any/all of the parts in which breath air flows, which are included in the device, upon which a pressure difference can be measured between a flow inlet and outlet or between a flow outlet and inlet. According to some embodiments, the variable air resistance structure may include a housing and may be anything through which breath air flows during the test, including the mask, the flow meter and/or any other element. According to some embodiments, the resistor housing is not limited to any shape and may be, for example: a cylinder, pipe, square pipe, polygonal pipe (triangle, hexagon, octagon and other types) or any other shape.

As used herein, the term “housing may refer to a structure configured to contain a flow modifying mechanism such as a resistor, and to allow a flow of gas therethrough.

According to some embodiments, the air flow resistor may include one or more shutters, flaps, valves, steel mesh, plastic mesh, perforated plates, slotted plates, orifices, porous materials, converging cones, pressed flexible pipes (reduced cross section), propeller, slots, angled pipes, long pipes, labyrinths or any combination thereof.

Flow meter 240 is configured to provide a volumetric measurement of the subject's breath flow rate. In operation, the subject's exhaled breath is collected by mask 230 and is transferred through tube 220 (arrow 222), through variable air resistance structure 210 then through to flow meter 240, and exhausted out (arrow 223). When the subject inhales, ambient air is sucked into flow meter 240 (arrow 225), passes through air resistance structure 210, then through tube 220 (arrow 224), and continues to the subject's mouth and/or nostril(s) through mask 230.

According to some embodiments, the pulmonary function testing system further includes a differential pressure sensor 270, which is configured, according to some embodiments, to measure the pressure difference of the pulmonary function testing system. According to some embodiments, in a case for which the resistance characteristics of the entire pulmonary function testing system are known for each air flow rate that is applied in a breath test and the atmospheric pressure is assumed to be around 1 Atm, the differential pressure sensor can be omitted from the system. According to some embodiments, modifying the assumed atmospheric pressure in accordance with altitude relative to sea level will increase accuracy. According to some embodiments, modifying the assumed atmospheric pressure in accordance with temperature will further increase accuracy. According to some embodiments, measuring atmospheric (ambient) pressure using ambient pressure sensor 272 will further increase accuracy.

According to some embodiments, the ambient pressure may be assumed/evaluated to be a certain pressure value or range, and the measurements and/or processing thereof are modified/calibrated accordingly. According to some embodiments, the ambient pressure may be evaluated based on the altitude and/or geographic location of the device at use. According to some embodiments, the ambient pressure may be provided by the user. According to some embodiments, the ambient pressure may be assumed to be in the range of 0.8-1.2 Atm.

Differential pressure sensor 270 has two pressure inputs, port 270a and port 270b between which it compares. Port 270a is shown connected to mask 230, but can be connected along the path between mask 230 and a point before (upstream of exhaled flow) air flow resistor 250. According to some embodiments, connecting port 270a as close as possible to the point of air entry (the mouth/nose) will increase accuracy. Port 270b should be open to the atmosphere and preferably not in proximity to any type of air flow or heat source. According to some embodiments, the differential pressure sensor may be replaced with one absolute or gauge pressure sensor and connect its input port between the mouth/nose and the air flow resistor.

According to some embodiments, the pulmonary function testing system further includes an ambient pressure sensor 272 configured to measure a pressure of a surrounding environment of the pulmonary function testing system. According to some embodiments, the pulmonary function testing system further includes an ambient temperature sensor 274 configured to measure a temperature of a surrounding environment of the pulmonary function testing system. According to some embodiments, the pulmonary function testing system further includes an internal temperature sensor 276 placed near air flow resistor 250, configured to measure a temperature of gasses passing through the pulmonary function testing system.

It is noted that, according to alternative embodiments, the subject's exhaled breath can also be transferred through a flow meter first and then through a variable air resistance structure. In other words, according to some embodiments, the flow meter may be placed before (upstream of exhaled flow) or after (downstream of exhaled flow) the air flow resistor. In case the flow meter is placed before the air flow resistor and a differential pressure sensor is used, its port must now be moved from before the resistor to be placed before the flow meter. However, if the flow resistance characteristics of the flow meter are known for every possible air flow rate that exists in a test, the port may be left before the resistor and a compensation for flow resistance may be made by a calculation of the known value.

The subject interface, which is configured to collect breath, is shown herein as a mask but, according to some embodiments, the subject interface can also be a tube or a cannula, a mouthpiece or any other device that facilitates the collection of essentially all exhaled breath gas and transfers it to the pulmonary function testing device and/or directs essentially all inhaled air through the entire flow path of the device from its inlet port to the mouth/nose.

Tubes 120/220 are shown having a certain length and diameter but, according to some embodiments, other appropriate lengths and diameters may be used. According to some embodiments, the subject interface (for example, the mask) may be directly connected to the pulmonary function testing device without the use of a tube and/or using more than one tube.

According to some embodiments, the pulmonary function testing system further includes a processing unit 280 configured to obtain measurement signals from flow meter 240 and pressure sensor 270, and is further configured to control resistance characteristic variation component 260 for varying an air resistance characteristics of air flow resistor 250. Processing unit 280 is further configured to analyze the measurements for providing test results. Processing unit 280 may include or be connected to a control interface 284, for providing a user of the device with control option over the pulmonary function testing system. Such control option may include a power switch, a test knob, a resistance knob, a test selector and the like. Processing unit 280 and/or other components of the pulmonary function testing system are provided with electric power for operation by an electric power source 286, such as an electric power socket, a battery or the like.

According to some embodiments, power source 286 may include a battery, an electricity outlet, an electric-power generation module, or any type of electrical, mechanical, chemical, potential energy source.

According to some embodiments, the pulmonary function testing system further includes a display 282, communicatively connected with processing unit 280. According to some embodiments, display 282 is configure to displaying pulmonary test results, configuring processing unit 280, displaying test progress and/or providing test instructions.

Optionally, according to some embodiments, processing unit 280 may be connected to an external computer 288. Connecting processing unit 280 to external computer 288 may be useful for operational, maintenance and/or logging purposes, or the like. The connection between processing unit 280 and external computer 288 may be a local wired connection, a local wireless connection, a remote wired connection and/or a remote wireless connection (such connections may include Bluetooth, Ethernet, USB, Wi-Fi and the like).

Reference is now made to FIG. 2b, which schematically illustrates a block diagram of a pulmonary function testing system, essentially as provided in FIG. 2a, wherein instead of mask 230, the system includes a mouthpiece 232 for providing respiratory gas from/to a user 290.

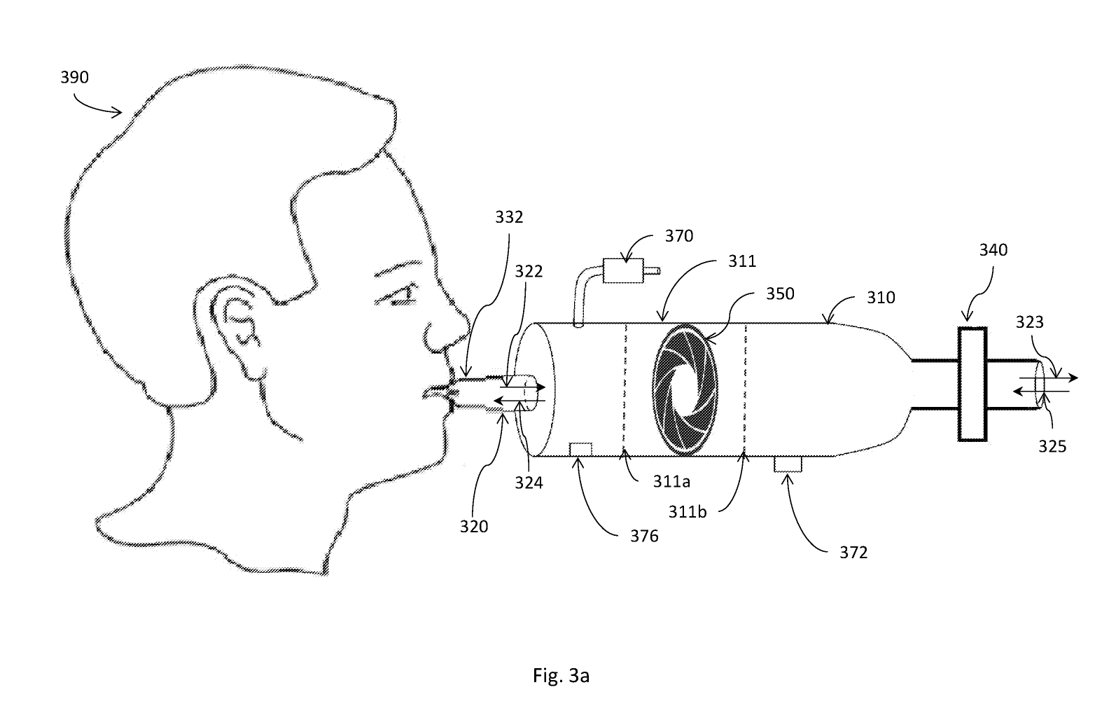

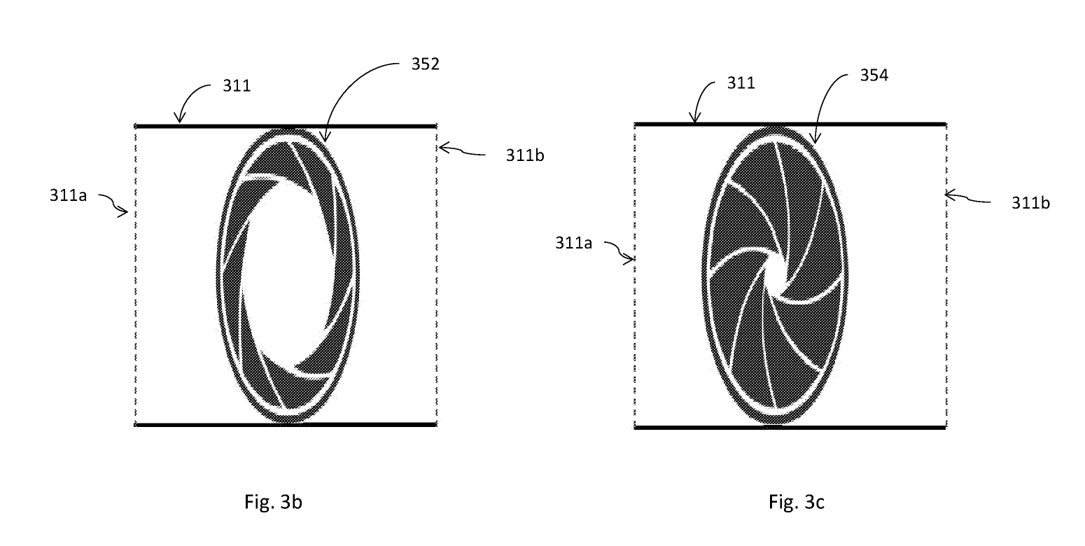

Reference is now made to FIG. 3a, which schematically illustrates a pulmonary function testing system with a pulmonary function testing device 310 having a variable air resistance structure, such as variable aperture shutter 350, within a flow-modification section 311 marked with first section marker 311a and second section marker 311b, according to some embodiments. Pulmonary function testing device 310 is in fluid flow (breath) connection with the respiratory system of a patient 390, through a respiratory opening thereof, facilitated by a user interface breath unit, such as mouthpiece 332, via a tube 320 such that exhaled gas (arrow 322) and inhaled gas (arrow 324) pass through tube 320, through variable aperture shutter 350, through a flow meter 340 and are exhausted to the environment (arrow 323) or sucked as intake from the environment (arrow 325) through a flow meter 340, through variable aperture shutter 350, through tube 320, then mouthpiece 332, and into the respiratory system of a patient 390. Flow meter 350 is configured to measure the flow of gas passing therethrough.

Additionally, the pulmonary function testing system includes a pressure sensor, such as differential pressure sensor 370, which is configured to measure a pressure difference between a location within pulmonary function testing device 310 and a location in the surrounding environment of the device (ambient). The location within pulmonary function testing device 310 is preferably between variable aperture shutter 350 and the patient's mouth (or mask placed on the mouth). During exhalation, exhaled gas (arrow 322) is forced through variable aperture shutter 350 and, as a result, pressure increases within pulmonary function testing device 310, and differential pressure sensor 370 measures a positive pressure difference between the location within pulmonary function testing device 310 and the location in the surrounding environment of the device.

During inhalation, inhaled gas (arrow 324) is sucked through variable aperture shutter 350 and, as a result, pressure drops within pulmonary function testing device 310, and differential pressure sensor 370 measures a negative pressure difference between the location within pulmonary function testing device 310 and the location in the surrounding environment of the device.

Optionally, according to some embodiments, pulmonary function testing device 310 may include a separate ambient pressure sensor 372 for measuring an ambient pressure in a surrounding environment of pulmonary function testing device 310.

Optionally, according to some embodiments, pulmonary function testing device 310 may include a temperature sensor 376 for measuring a temperature of gas within pulmonary function testing device 310.

The resistance of variable aperture shutter 350 may be controlled by varying the aperture thereof.

Reference is now made to FIG. 3b, which schematically illustrates a flow modification section 311 of pulmonary function testing device 310 as described in FIG. 3a, marked with first section marker 311a and second section marker 311b, wherein variable aperture shutter 352 is at a wide aperture position, in which the aperture of variable aperture shutter 352 is increased. An increased aperture may provide a low resistance characteristic for variable aperture shutter 352.

Reference is now made to FIG. 3c, which schematically illustrates a flow modification section 311 of pulmonary function testing device 310 as described in FIG. 3a, marked with first section marker 311a and second section marker 311b, wherein variable aperture shutter 354 is at a narrow aperture position, in which the aperture of variable aperture shutter 354 is decreased/reduced. A decreased/reduced aperture may provide a high resistance characteristic for variable aperture shutter 354.

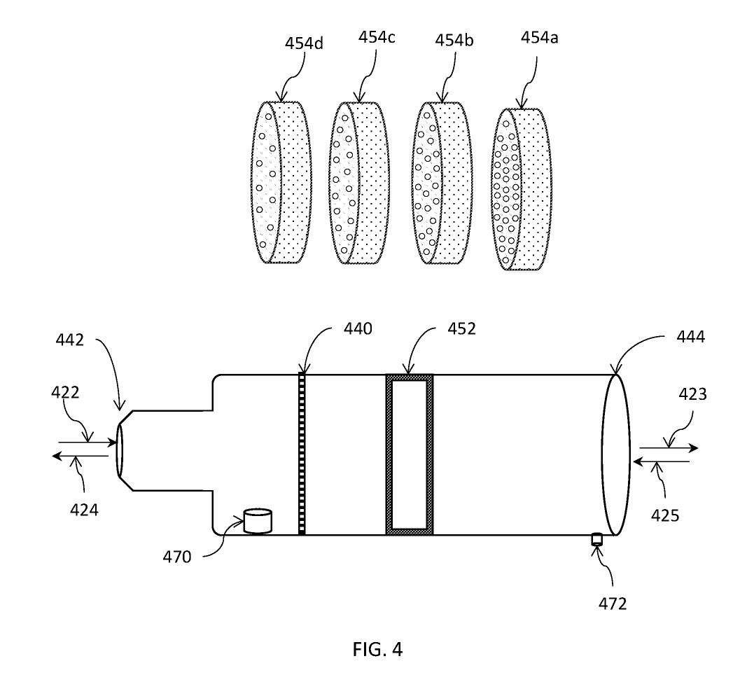

Reference is now made to FIG. 4, which schematically illustrates a pulmonary function testing device having multiple air resistors, such as multiple perforated discs 454a, 454b, 454c, and 454d, varying in the resistance characteristics therebetween, and a receptacle 452 configured to hold/host at least one of multiple perforated discs 454a, 454b, 454c, and 454d. According to some embodiments, one of multiple perforated discs 454a, 454b, 454c, or 454d, may be manually selected and inserted in receptacle 452 for achieving desired air-flow characteristics.

According to some embodiments, the function testing device includes a flow meter 440 for measuring the flow of gasses therein, an internal pressure sensor 470 for measuring the pressure within the function testing device, and an ambient pressure sensor 472 for measuring the pressure of a surrounding environment of the function testing device.

During exhalation, exhaled respiratory gas (arrow 422) is introduced to a respiration aperture 442 in the function testing device, then the gas passes through flow meter 440 and through receptacle 452 having a perforated disk therein, then the gas is exhausted (arrow 423) from the function testing device through external aperture 444.

During inhalation, surrounding gas is sucked (arrow 425) into the function testing device through external aperture 444 and forced through receptacle 452 having a perforated disk therein, then through flow meter 440 and to the respiratory system of a user (not shown) through respiration aperture 442 (arrow 424).

According to some embodiments, signals from the flow meter, pressure sensor(s) and/or temperature sensor(s) are obtained by the controller, which performs a computational analysis between corresponding intervals of a first representative respiration and a second representative respiration to derive test results.

As used herein, the terms corresponding respiratory phases, and corresponding intervals, refer to points or intervals (ranges) in two respiratory cycles, being identified to have similar properties or being related to a common value/measurement. For example, corresponding respiratory phases and/or corresponding respiratory intervals may refer to two points and/or intervals in two respiratory cycles (or representative cycles) having a certain volume approximation/measurement, or falling within a certain range of approximation/measurement of a parameter, such as a volume, ratio of time within the cycle, time lapsed from last landmark (such as end of breath) or the like. According to some embodiments, a range may be selected for providing tolerance in the selection of the phases and/or intervals.

As used herein, the term “representative cycle” or “representative respiratory cycle” may refer to an aggregation of at least two cycles to generate one cycle, signifying data related to the aggregated cycles. According to some embodiments, the aggregation may include averaging, weighted averaging, error/noise detection, smoothing or the like or any combination thereof.

According to some embodiments, the computational analysis per interval comprises deriving the airflow measurements. According to some embodiments, the computational analysis takes into consideration the temperature within the device, the temperature outside the device, the pressure within the device and/or the pressure outside the device and/or the like, or any combination thereof. According to some embodiments, the temperature and/or pressure measurements are used for altering the airflow measurements. According to some embodiments, the temperature and/or pressure measurements are used to increase the accuracy of the airflow measurements.

According to some embodiments, the computational analysis comprises time derivation, integration, frequency analysis, and the like, of the airflow and/or pressure signal at least in some intervals.

According to some embodiments, the sensory signals are delivered to an analysis unit to perform the computational analysis and/or accuracy/normalization/alteration and/or other computational tasks.

According to some embodiments, one or more airflow resistance structures may be porous, slotted, or otherwise semipermeable rigid, semi-rigid, or flexible devices that may block or restrain a substantial portion of a chamber through which respiratory gasses may flow, thereby limiting or restricting airflow by a measurable amount. Such an airflow resistance structure may have a desirable shape (such as disc, flat square, curved facings, or other) provided the device will restrict airflow through the spirometry device by a measurable amount. For illustrative purposes, porous flat discs are introduced, although other shapes may also fit the disclosure.

Airflow resistors may include, for example: a flap, a valve, a steel mesh, a perforated plate, a plastic mesh, a slotted plate, an orifice, a porous material, a converging cone, a pressed flexible pipe (reduced cross section area), a propeller, a slot, a pipe/hose, an angled pipe/hose, a labyrinth or the like.

According to some embodiments, the term “resistor” may be interchangeable with the terms “restrictor”, “obstructer”, or the like, and refers to a structure configured to affect the flow of gas therethrough or in the vicinity thereof.

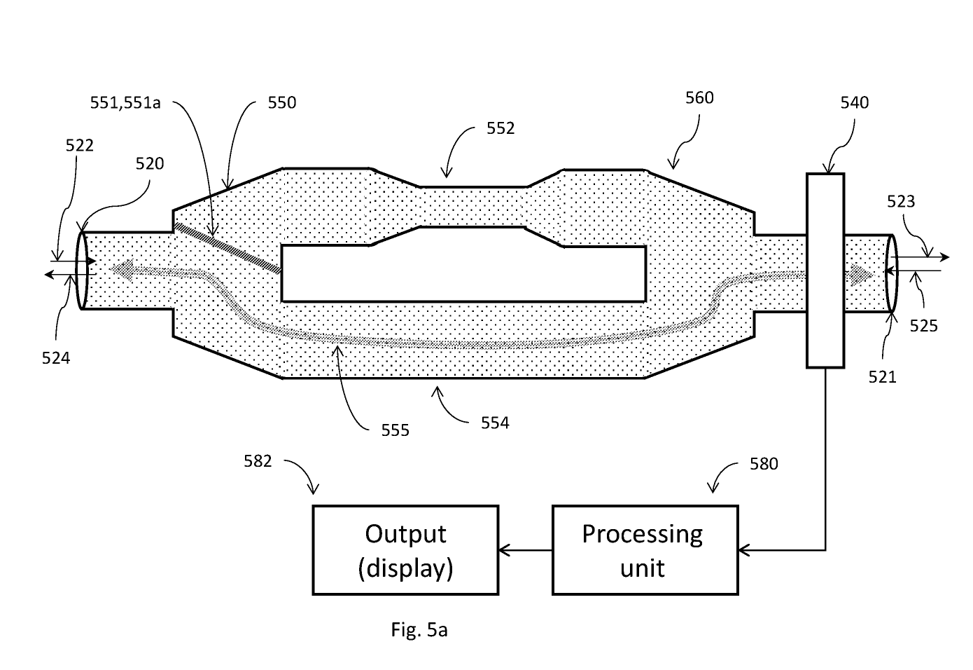

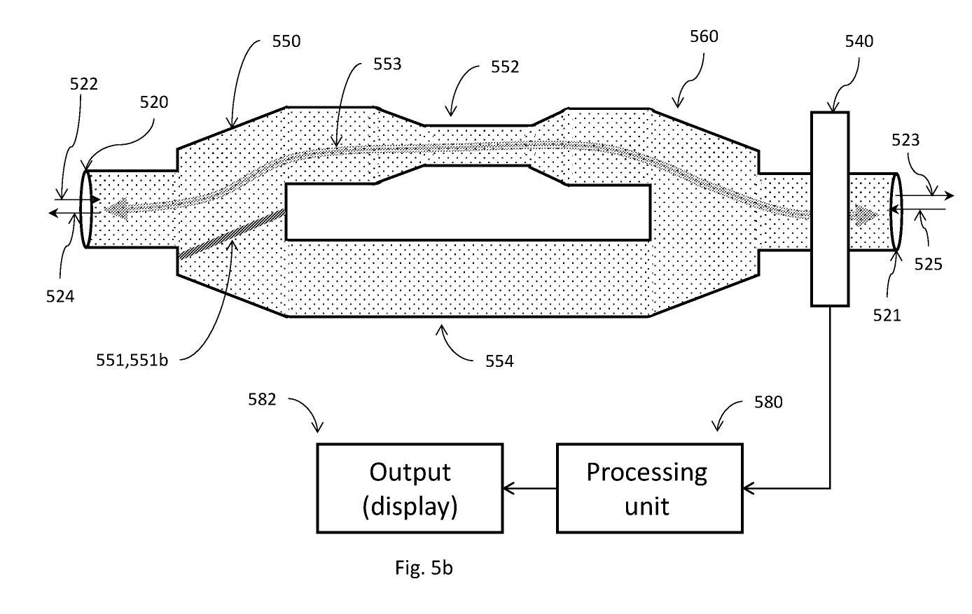

Reference is now made to FIG. 5a, which schematically illustrates a pulmonary function testing device included with a flow valve 550 at a first position, according to some embodiments. According to some embodiments, the pulmonary function testing device includes an inlet aperture 520 through which respiratory gas flow is permitted to and from the device (arrows 522 and 524 respectively), and an outlet aperture 521 for exhausting respiratory gas from the device and inletting gas to the device (arrows 523 and 525 respectively). According to some embodiments, the device further includes a flow modulator, such as valve structure 550 including a valve-flap 551, for controllably directing the flow of the gas within the device either through a first conduit 552 or through a second conduit 554. According to some embodiments, when valve-flap 551 is in a first position 551a, the gas flow is directed via first conduit 554 having a first set of characteristics (arrow 555). The flow of gas then passes through a funnel 560 for directing the gas flow through either conduit (552 or 554) to a flow-meter 540, which provides signals indicative of a flow of gas therethrough to a processing unit 580 for further analysis of at least a first respiratory cycle, or portions thereof. Afterwards, the flow of gas passes through outlet aperture 521. Processing unit 580 is configured to provide information indicative of the measurements and/or status of the test to an output device, such as display 582 to be displayed to a user and/or an operator.

Reference is now made to FIG. 5b, which schematically illustrates a pulmonary function testing device included with a flow modulator, such as valve structure 550, essentially as provided in FIG. 5a, at a first position, according to some embodiments. According to some embodiments, when valve-flap 551 is at a second position 551b, the gas flow is directed via second conduit 552 having a second set of characteristics (arrow 553). The flow of gas then passes through a funnel 560 for directing the gas flow through either conduit (552 or 554) to the flow-meter 540, which provides signals indicative of a flow of gas therethrough to a processing unit 580 for further analysis of at least a second respiratory cycle, or portions thereof. Afterwards, the flow of gas passes through outlet aperture 521.

According to some embodiments, a pulmonary function test is initiated by selecting a first resistance characteristic, then a subject breathes through a mask and a first representative respiration is derived by an analysis unit (controller). Then a second resistance characteristic is selected and the subject breathes through the mask and a second representative respiration is derived by the analysis unit (controller).

According to some embodiments, an air resistance structure may include multiple porous discs having varying multiple air resistance characteristics. According to some embodiments, the multiple resistance characteristics are achieved by having varying density of the pores between the discs. According to some embodiments, the multiple resistance characteristics are achieved by having varying size of the pores between the discs. According to some embodiments, an air resistance device comprises a porous medium, configured to facilitate restricted airflow therethrough. According to some embodiments, the air resistance device comprises porous discs. According to some embodiments, the air resistance device comprises porous sheets.

According to some embodiments, the pores of the porous medium are uniformly distributed. According to some embodiments, the pores of the porous medium are non-uniformly distributed.

According to some embodiments, each porous medium is characterized by pores having an approximately similar pore size per medium. According to some embodiments, the pore size may determine an air resistance characteristic of a certain porous medium. According to some embodiments, a porous medium having larger pore density is characterized by a lower air resistance characteristic than a porous medium having a smaller pore density.

According to some embodiments, each porous medium is characterized by a predetermined pore density. According to some embodiments, the pore density dictates and/or determines the air resistance characteristic of each of the porous mediums. According to some embodiments, a porous medium having a higher pore density is characterized by a lower air resistance characteristic than a porous medium having a lower pore density.

According to some embodiments, the air resistance porous mediums are characterized by approximately the same pore density and pore size. According to some embodiments the porous mediums may vary from one another in size and/or shape. According to some embodiments, the size and/or shape of a porous medium determine the air resistance characteristic of the medium. According to some embodiments, the porous mediums may vary from one another in thickness. According to some embodiments, a thicker porous medium may be characterized by a higher air resistance characteristic than a slimmer medium.

According to some embodiments, an air resistance structure may comprise two or more selectable air resistance characteristics. According to some embodiments, an air resistance structure may comprise 2 different selectable air resistance characteristics. According to some embodiments, an air resistance structure may comprise 3 different selectable air resistance characteristics. According to some embodiments, an air resistance structure may comprise at least 10 different selectable air resistance characteristics. According to some embodiments, an air resistance structure may comprise at least 40 different selectable air resistance characteristics. According to some embodiments, an air resistance structure may comprise at least 100 different selectable air resistance characteristics.

According to some embodiments, a pulmonary function test is performed by breathing through the pulmonary function test device two or more times. According to some embodiments, a pulmonary function test is performed by breathing through the device at least 3 times. According to some embodiments, a pulmonary function test is performed by breathing through the device multiple times, at least some breaths or portions thereof are forced through a first air resistance characteristic and at least some other breaths or portions thereof are performed through a second air resistance characteristic.

According to some embodiments, a pulmonary function test is performed by breathing through the device multiple times, at least some breaths or portions thereof are forced through a first air resistance characteristic, at least some breaths or portions thereof are performed through a second air resistance characteristic, at least some breaths or portions thereof are performed through a third air resistance characteristic. According to some embodiments, at least some other breaths or portions thereof are performed through a forth air resistance characteristic. According to some embodiments, at least some other breaths or portions thereof are performed through a fifth air resistance characteristic. According to some embodiments, at least some other breaths or portions thereof are performed through a sixth air resistance characteristic.

As used herein, the term “air inlet” and the term “respiration aperture” may refer to an opening in the pulmonary function test device or system, through which a subject may inhale and/or exhale to provide respiratory gasses to the chamber and air resistance structure. According to some embodiments, an air inlet may be a mask. According to some embodiments, an air inlet may be integrally formed with a mask. According to some embodiments, an air inlet may be connected to a mask using a tube system. According to some embodiments, an air inlet is a nozzle. According to some embodiments, an air inlet is an opening at a proximal end of the device.

As used herein, the term “airflow exit”, the term “outlet” and the term “external aperture” are interchangeable, and may refer to an opening in the pulmonary function test device or system through which exhaled respiratory gasses may pass from the chamber to a surrounding environment and inhaled respiratory gasses may pass from a surrounding environment to the chamber. According to some embodiments, an airflow exit is an opening at a distal end of the air chamber of the pulmonary function test device.

As used herein, the term “air chamber” may refer to a structure configured to facilitate a flow of respiratory gasses from an air inlet to an airflow exit. According to some embodiments, an air chamber is further configured to at least partially and/or temporarily host an air resistance structure. According to some embodiments, an air chamber is further configured to at least partially and/or temporarily host at least one sensor such as an airflow sensor, a pressure sensor, a temperature sensor and the like, and/or a plurality and/or any combination thereof.

As used herein, the term “air resistance structure” may refer to any structure or device that is configured to restrain or limit airflow therethrough. According to some embodiments, restraining or limiting airflow provides changes in pressure values and/or flow rate compared to unrestrained airflow.

As used herein, the term “controllable air resistance structure” may refer to a structure configured to act as an air resisting agent by disrupting, partially obstructing, limiting, modifying and/or modulating an airflow therethrough, thereby developing a pressure gradient between a first side and a second side thereof and/or a difference in the flow characteristics of the airflow compared with an absence thereof, wherein the amount of the disruption, obstruction, limiting, modifying and/or modulating of an airflow may be selected from a predetermined set of air-resistance characteristics and/or a continuum of air resistance characteristics.

As used herein, the terms “display” and “monitor” are interchangeable. According to some embodiments, a display or a monitor are configured to provide visual and/or audial indications to a user.

According to some embodiments, a first respiration cycle may include two or more respiration cycles respired through a first air resistance characteristic. According to some embodiments, a first respiration cycle may include a set of respiration cycles, wherein the set of respiration cycles comprises successive respiration cycles. According to some embodiments, a first respiration cycle may include multiple respiration cycles respired through a first air resistance characteristic. According to some embodiments, a representative “first-cycle” is derived from the multiple respiration cycles. According to some embodiments, the representative “first-cycle” is derived from the multiple respiration cycles by averaging the measurements of the multiple respiration cycles. According to some embodiments, the representative “first-cycle” is derived from the multiple respiratory cycles by a weighted averaging of the measurements of the multiple respiratory cycles such that converging respirations are given a higher weight in the weighted average than non-converging respiratory cycles. According to some embodiments, the representative “first-cycle” is derived from the multiple respiration cycles by eliminating measurements of respiration cycles that fall out of a normal behavior of a respiration.

According to some embodiments, a second respiration cycle may include one or more respiration cycles respired through a second air resistance characteristic. According to some embodiments, a second respiration cycle may include a set of respiration cycles, wherein the set of respiration cycles comprises successive respiration cycles. According to some embodiments, a second respiration cycle may include multiple respiration cycles respired through a second air resistance characteristic. According to some embodiments, a representative “second-cycle” is derived from the multiple respiration cycles. According to some embodiments, the representative “second-cycle” is derived from the multiple respiration cycles by averaging the measurements of the multiple respiration cycles. According to some embodiments, the representative “second-cycle” is derived from the multiple respiratory cycles by a weighted averaging of the measurements of the multiple respiratory cycles such that converging respirations are given a higher weight in the weighted average than non-converging respiratory cycles. According to some embodiments, the representative “second-cycle” is derived from the multiple respiration cycles by eliminating measurements of respiration cycles that fall out of a normal behavior of a respiration.

According to some embodiments, a normal behavior of a respiration is a global parameter including global respiratory characteristic norms and margins. According to some embodiments, a normal behavior of respiration is associated with a special group or section of the population; such a group may be categorized by gender, age, health condition and others. According to some embodiments, a normal behavior of a respiration is a private parameter derived from the multiple respiration cycles of a specific subject.

According to some embodiments, a first air resistance characteristic and a second air resistance characteristic are selected from multiple selectable distinct air resistance characteristics provided by the air resistance structure. According to some embodiments, the multiple selectable air resistance characteristics include at least two distinct air resistance characteristics. According to some embodiments, the multiple selectable air resistance characteristics include at least three distinct air resistance characteristics. According to some embodiments, the multiple selectable air resistance characteristics include more than 10 distinct air resistance characteristics.

According to some embodiments, the air resistance structure is configured to provide controllable selection of resistance characteristics from a discrete predetermined set of resistance characteristics. According to some embodiments, the air resistance structure is configured to provide a controllable selection of resistance characteristics from a continuous range of resistance characteristics. According to some embodiments, the multiple selectable air resistance characteristics are characteristics selectable from a continuum of resistance characteristics.

According to some embodiments, the device may include an airflow sensor configured to measure the flow of respiration gasses respired through the air resistance structure having a selected air resistance characteristic. According to some embodiments the airflow sensor provides volumetric air flow measurements. According to some embodiments, the airflow sensor is configured to measure a flow of air in units of volume per time.

According to some embodiments, the airflow sensor is an air flow meter. According to some embodiments, the airflow sensor is a mass air flow sensor. According to some embodiments, the airflow sensor is a “moving vane meter” volume air flow meter. According to some embodiments, the airflow sensor is a “hot wire sensor”. According to some embodiments, the airflow sensor is a “Coldwire sensor”. According to some embodiments, the airflow sensor is a “Kármán vortex sensor”. According to some embodiments, the airflow sensor is a “membrane sensor”.

According to some embodiments, a flow meter, or an airflow sensor may include one or more of: Moving wings, Micro turbine, Rotameter, Orifice, Venturi, Shutters, Grid etc., Filament/hot wire, Cold wire, Hot film, Acoustic Doppler, Optical Doppler, Doppler electromagnetic, Charged body Flow, Charging body Flow and the like.

According to some embodiments, the device may include a temperature sensor. According to some embodiments, the temperature sensor is configured to provide temperature measurements of the respiratory gasses before passing through the air resistance structure. According to some embodiments, the temperature sensor is configured to provide temperature measurements of the respiratory gasses after passing through the air resistance structure.

According to some embodiments, the device may include a temperature sensor for measuring the temperature of an environment near or surrounding the device.

According to some embodiments, the device may include a pressure sensor for measuring air pressure within the device.

According to some embodiments, the device may include a pressure sensor for measuring an ambient air pressure of an environment surrounding the device.

According to some embodiments, the devise derives accurate air flow measurements by taking into consideration measurements of temperature of air before passing through the air resistance structure, temperature of air after passing through the air resistance structure, pressure of air before passing through the air resistance structure, pressure of air after passing through the air resistance structure, temperature of a surrounding environment of the device and/or pressure of a surrounding environment of the device.

According to some embodiments, the devise provides a representative “first-cycle” and a representative “second-cycle”, associated with a first air resistance characteristic and a second air resistance characteristic, respectively.

According to some embodiments, the device compares the first cycle (or representative “first cycle”) with the second cycle (or representative “second cycle”), and analyzes the differences between the cycles. According to some embodiments, the device compares the flow values of the first cycle with the flow values of the second cycle at corresponding respiratory phases. According to some embodiments, the device compares the gradient of the flow values of the first cycle with the gradient of the flow values of the second cycles at corresponding respiratory phases.

As used herein, the term “respiratory cycle” refers to the inspiration and expiration of respiratory gasses. The term may also be used in the context of any measurement or value related to the inspiration and expiration of respiratory gasses.

As used herein, the term “respiratory phases” refers to any index that may provide a meaningful dissection or partitioning of a respiratory cycle. One example of a course partitioning is the direction of respiratory flow, and the phases in this case would be an inspiration phase and an expiration phase. One example of a fine partitioning is the estimated or measured volume of the lungs and/or the flow of respiratory gasses or the direction thereof.

According to some embodiments, the device compares the first and second cycles at corresponding respiratory phases. According to some embodiments, the device compares the first and second cycles at multiple intervals of the respiratory phases, such that the values of the first cycle at each interval are compared with the values of the second cycle at a corresponding interval.

The different resistance characteristics associated with the first representative cycle and the second representative cycle result in a different behavior of flow as a function of volume. According to some embodiments, this difference in behavior is analyzed per phase interval, and relevant respiratory parameters are derived from the analysis.

As used herein, the term “respiratory parameters” refers to parameters indicative of a respiratory function or state. Such a parameter may vary between different respiratory phases, or be constant throughout the entire respiratory cycle or parts thereof. The parameters may be discrete or continuous.

A respiratory related parameter may include:
An airway resistance value of a subject, as a constant approximation over a respiratory cycle, or varying across various respiratory phases.
A lung compliance of a patient, as a constant approximation over a respiratory cycle, or varying across various respiratory phases.

According to some embodiments, the device is configured to evaluate a lung compliance of the patient at various respiratory phases.

As used herein, according to some embodiments, the terms respiratory parameters” and “respiratory related parameters” may be interchangeable.

After obtaining a representative first cycle associated with selecting an air resistance characteristic of R1, and a representative second cycle associated with selecting an air resistance characteristic of R2, the following steps are utilized: According to some embodiments, a representative respiratory cycle can be constructed from multiple breath cycles or parts/portions thereof.

According to some embodiments, pulmonary compliance is calculated using the following equation, where ΔV is the change in volume, and ΔP is the change in alveolar (intrapleural pressure may also be used) pressure:

$$Compliance = \frac{Δ V}{Δ P}$$

For example, if a patient inhales 500 mL of air from a spirometer with an intrapleural pressure before inspiration of −5 cm H₂O and −10 cm H₂O at the end of inspiration. Then:

$$Compliance = \frac{Δ V}{Δ P} = \frac{.5 L}{( - 5 cm {H}_{2} O - ( - 10 cm {H}_{2} O ) )} = \frac{.5 L}{5 cm {H}_{2} O} = 0.1 L \times cm {H}_{2} {O}^{- 1}$$

### Static Compliance (Cₛₜₐₜ):

Static compliance represents pulmonary compliance during periods without gas flow, such as during an inspiratory pause. It can be calculated with the formula (wherein Pₚₗₐₜ designates plateau pressure):

$${C}_{stat} = \frac{{V}_{T}}{{P}_{plat} - PEEP}$$

Dynamic Compliance (C_{dyn}):

Dynamic compliance represents pulmonary compliance during periods of gas flow, such as during active inspiration. Dynamic compliance is always less than or equal to static lung compliance. It can be calculated using the following equation, where C_{dyn}=Dynamic compliance; V_{T}=tidal volume; PIP=Peak inspiratory pressure (the maximum pressure during inspiration); PEEP=Positive End Expiratory Pressure:

$${C}_{dyn} = \frac{{V}_{T}}{PIP - PEEP}$$

The slope of the pressure-volume curve, or the volume change per unit pressure change, is known as the compliance. In the normal range (expanding pressure of about −5 to −10 cm water) the lung is remarkably distensible or very compliant. The compliance of the human lung is about 200 ml/cm H₂O. However, at high expanding pressures, the lung is stiffer, and its compliance is smaller. A reduced compliance is caused by an increase of fibrous tissue in the lung (pulmonary fibrosis). In addition, compliance is reduced by alveolar edema, which prevents the inflation of some alveoli. Compliance also falls if the lung remains unventilated for a long period, especially if its volume is low. This may be partly caused by atelectasis (collapse) of some units, but increases in surface tension also occur. Compliance is also reduced somewhat if the pulmonary venous pressure is increased and the lung becomes engorged with blood. An increased compliance occurs in pulmonary emphysema and in the normal aging lung. In both instances, an alteration in the elastic tissue in the lung is probably responsible. Increased compliance also occurs during an asthma attack, but the reason is unclear. The compliance of a lung depends on its size. Clearly, the change in volume per unit change of pressure will be larger for a human lung than, say, a mouse lung. For this reason, the compliance per unit volume of lung, or specific compliance, is sometimes measured if we wish to draw conclusions about the intrinsic elastic properties of the lung tissue.

According to some embodiments, the selected first and second air resistance characteristics are alternated during a respiration cycle. According to some embodiments, the first and second air resistance characteristics are alternated at least twice within a relevant respiratory phase interval.

According to some embodiments, the air resistance characteristics are selected such that the normal respiration of the subject is not disrupted by the resistance characteristics. According to some embodiments, the desired selected air resistance characteristics are lower than 10% of an airway resistance of the subject. According to some embodiments, the desired selected characteristics are no more than 15% of an airway resistance characteristic of the subject.

According to some embodiments, the difference between the first air resistance characteristic and the second air resistance characteristic is at least 1%.

According to some embodiments, the difference between the first air resistance characteristic and the second air resistance characteristic is in the range of 0.1% to 20%. According to some embodiments, the difference between the first air resistance characteristic and the second air resistance characteristic is in the range of 1% to 10%. According to some embodiments, the difference between the first air resistance characteristic and the second air resistance characteristic is in the range of 2% to 5%.

The airway resistance characteristic of the subject may not be available prior to using the device, therefore an estimation of allowable or desirable air resistance characteristics is estimated based on the health condition of the subject, age, weight, height, gender and or other conditions or parameters.

According to some embodiments, for some respiratory related parameters, there exists a desired range of values that are considered acceptable, and a patient whose evaluated respiratory related parameter falls within that desired range of values will be considered healthy (at least with a specific condition related to that respiratory parameter). Alternatively, if the evaluation of the respiratory parameters is below or above that desired range, it might be indicative of a respiratory related condition that might call for further attention.

According to some embodiments, the device provides the evaluation of the respiratory related parameter with error margins. The evaluation and the error margins provide an evaluated range for the respiratory related parameter.

According to some embodiments, if the evaluated range falls completely within the desired range, the device may declare that the test is done and that the results fall within the desired range. In other cases, when the evaluated range falls completely below or completely above the desired margins, the device may declare that the test is done and that the results fall outside the desired range.

According to some embodiments, a reliability index is derived for the results of the tests. A reliability index may provide indication to a healthcare giver on whether or not further testing is required.

According to some embodiments, a reliability index is a binary reliability index, indicating whether the results are reliable or not.

According to some embodiments, a reliability index is a grade reliability index, providing a grade indicating how accurate the test was.

According to some embodiments, the device may include a monitor for providing textual, graphical and/or audial indication to the subject and/or healthcare provider regarding the test itself or regarding relevant respiratory related parameters.

According to some embodiments, the monitor is configured to display a graph of the relevant respiratory related parameter.

According to some embodiments, the device is configured to receive information from a user or a healthcare provider regarding the health condition of the user/subject and/or other relevant test information such as age, gender, weight, height and other relevant information.

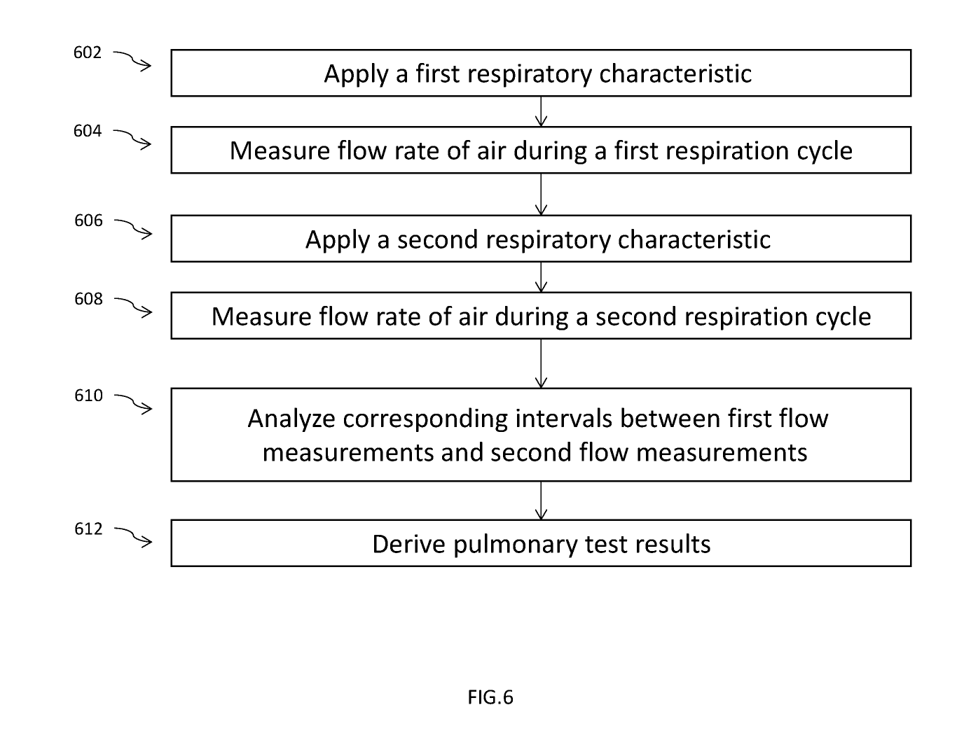

Reference is now made to FIG. 6, which illustrates a flow chart of a method for testing pulmonary function using a variable air resistance structure, according to some embodiments. The method begins by applying a first respiratory characteristic (step 602), then measuring the flow rate of air during at least a first respiration cycle (step 604) or substantial part thereof, then applying a second resistance characteristic (step 606) and measuring the flow rate of air during at least a second respiration cycle (step 608) or substantial part thereof, then analyzing corresponding intervals between first flow measurements and second flow measurements (step 610), and deriving pulmonary test results (step 612).

According to some embodiments, the selection of air resistance characteristics is recursive. According to some embodiments, the selection of air resistance characteristics is dynamic.

According to some embodiments, for conducting a test with the device, two or more resistance characteristics are required. Furthermore, all or a major portion of the breath gasses being inhaled and/or exhaled is required to pass through the device.

According to some embodiments, the resistance characteristic of a flow resistor correlates with the flow rate through the resistance structure; therefore the characteristic may be defined either by physically altering the resistor structure or by changing the rate of fluid (breath gases) flow, wherein rate may be to volume gas per unit time, or mass gas per unit time or the like. According to some embodiments, the two different resistance characteristics of the flow resistor(s) may be defined in order to conduct the pulmonary function test, the characteristics are such that the subject can breathe through the test device without being asphyxiated or suffocated and the corresponding pressure sensor can register the difference in pressure between the two relevant points of the flow path.

According to some embodiments, to increase test accuracy, the resistance characteristics during the test are selected to be between a minimum value in which the relevant pressure sensor may register the difference in pressure between the two points of the flow path to 0.2 (or 20%) of the subjects' airway resistance characteristic. A higher value than 0.2 of the subjects' airway resistance value may also be used in order to yield correct test results.

According to some embodiments, a resistance characteristic range is calculated by setting the device resistance to a preliminary resistance characteristic being the minimal possible characteristic or entering a predefined characteristic while monitoring the following parameters either by direct sensor measurement or computational analysis for the duration of at least 1 breath cycle, (multiple breath cycles may be beneficial): Breath flow rate (either mass or volumetric) or flow speed, Breath cadence (rate) and/or mouth pressure, wherein the mouth pressure may be measured as the gas pressure in the mask or mouthpiece used by the user.

Afterwards, proceed by averaging the parameters at the preliminary resistance value, and setting each average as a reference point for each corresponding parameter. The resistance value may then be gradually raised/increased in order to detect a change in the above mentioned reference parameters. A change may include an elevated or reduced flow rate, an elevated or reduced breath cadence, an elevated mouth pressure during exhalation and/or a reduced mouth pressure during inhalation.

According to some embodiments, if a change of 1% or more, such as 5%, 6%, 7%, 8%, 10%, 15%, 50%, 100%, 200% etc. in the reference parameters is identified, the resistance value may or may not then be lowered to a value that reduces the change and the parameters return to their previous state. This value may be defined as ‘MAX’. A change of less than 1% such as 0.9%, 0.8%, 0.5%, 0.1%, 0.05% etc. may also be used for this process although it may yield lower accuracy in the measurements. The range of resistance values may then be defined to be from a minimum value in which the pressure sensor can register the difference in pressure between the two relevant points of the flow path and a maximum value herein defined as MAX.

According to some embodiments, an exemplary test procedure may be as follows:

A first resistance value is selected, and a first pulmonary function test is conducted while measuring at least a substantial part of a breath cycle. (According to some embodiments, more breath cycles may be beneficial for accurate results such as 2, 3, 5, 10 or more breaths) The relevant data may be recorded by direct sensor measurement and/or computational analysis based on measurements.

A second resistance value is selected, the second resistance value is different from the first value, then a second pulmonary function test is conducted with measurements of at least a substantial part of a breath cycle, (According to some embodiments, more breath cycles may be beneficial for accurate results such as 2, 3, 5, 10 or more breaths). The relevant data may be recorded by direct sensor measurement and/or computational analysis based on measurements.

The values recorded from the first and second pulmonary function tests are used to calculate at least one of the following parameters by solving either two equations with 2 variables or 3 equations with 2 variables: Airway resistance, Alveolar pressure and Lung compliance.

According to some embodiments, for calculating the airway resistance and/or alveolar pressure for a set of momentarily (interval/phase) measured parameters, the following two equations containing two variables is solved:

$${R}_{1} + {R}_{aw} = \frac{( {P}_{alv} - {P}_{amb} )}{\dot{V}}$$

$${R}_{2} + {R}_{aw} = \frac{( {P}_{alv} - {P}_{amb} )}{\dot{V}}$$

R₁ is the momentary resistance value of resistance characteristic number 1.
R₂ is the momentary resistance value of resistance characteristic number 2.
R_{aw} is the momentary resistance value of the airway/respiratory system.
Pₐₗᵥ is alveolar pressure.
\dot{V} is volumetric flow rate.

According to some embodiments, for calculating lung compliance for two sets of momentarily measured parameters, the following equation may be solved after solving the two equations mentioned earlier for airway resistance and/or alveolar pressure:

$${C}_{res} = \frac{Δ V}{Δ {P}_{alv}}$$

Cᵣₑₛ is the compliance of the respiratory system.
ΔV is the difference in volume between two measured points.
ΔPₐₗᵥ is the difference in pressure between two measured points.

Two pulmonary function tests are done for calculating the results; however, more tests may be done for raising accuracy, if required. As an example, several tests may be done in order to attain an average value for various phases of the subjects' breath cycle.

According to some embodiments, the flow rate measured value is utilized for calculating the airway resistance and/or alveolar pressure. According to some embodiments, measuring additional parameters may improve the accuracy of the test: a pressure difference between mask and ambient (or other locations within the device and ambient) temperature of air within the device, ambient pressure, humidity within the device, ambient humidity.

According to some embodiments, the ambient pressure sensor is located in the vicinity of the test (for example in the same room as the device). Preferably, the ambient pressure sensor is located away from effects that may alter the measurements; such effects may include a flow of air, a heat source, a cold air source and the like.

According to some embodiments, the device may further include one or more temperature sensors. When one temperature is used, it may be placed before the resistor and adjacent to it. When more than one temperature sensor is used, they may be located close to the mouth and close to the flow meter or before and after any part of the flow path that presents head loss (additional resistance).

According to some embodiments, the device may further include a temperature control device configured to control the temperature of air within the device and/or elements of the device. Advantageously, using a temperature control device may improve the accuracy by maintaining an identical temperature at the mouth and at the flow meter and thereby maintain a relatively constant air flow rate.

According to some embodiments, there is provided an automated method, in which the selected resistors (which determine the resistance value for the first, second and/or additional values) are automatically selected by the device by changing the resistance value.

According to some embodiments, the resistor may be set to the smallest resistance value by the device, and the first test will be performed using the same procedure explained above, while the values are changed automatically by the device.

According to some embodiments, some parameters, such as the subject's age, sex, weight, height, prior known respiratory illnesses and/or the like, may be entered into the device via a user interface (user interface breath unit), then the device may select an appropriate first resistance value and proceed with the same procedure explained above, while the resistance characteristics are changed automatically by the device.

According to some embodiments, the user interface breath unit may include, a nozzle, a mask, a mouthpiece, a tube or other structures configured to facilitate obtaining and providing respiratory gas from and to the user, respectively, through the device. According to some embodiments, there is provided a pulmonary function testing device having an inlet aperture/orifice configured to provide respiratory gas from a user to the device during exhale, and from the device to the user during inhale, an outlet/exhaust aperture/orifice configured to exhaust respiratory gas from a device to the surroundings thereof (ambient air) during exhale, and to provide gas from the surrounding of the device (ambient air) to the device during inhale, and the device further comprises a variable/adjustable airflow resistance mechanism including at least one air flow resistor configured to resist the flow of respiratory through the device. According to some embodiments, the devices may further include a flow sensor, configured to measure the flow of gas within the device. Alternatively, according to some embodiments, the devices may further include a pressure sensor, configured to measure the pressure within the device at one point, at two points or more.

According to some embodiments, there is provided a pulmonary function testing device having an inlet aperture/orifice configured to provide respiratory gas from a user to the device during exhale, and from the device to the user during inhale, an outlet/exhaust aperture/orifice configured to exhaust respiratory gas from a device to the surroundings thereof (ambient air) during exhale, and to provide gas from the surrounding of the device (ambient air) to the device during inhale, and the device further has a variable/adjustable air flow altering mechanism configured to modify (increase/decrease) the flow of gas within the device, for example by rotation of a propeller. According to some embodiments, the devices may further include a flow sensor, configured to measure the flow of gas within the device. Alternatively, according to some embodiments, the devices may further include a pressure sensor, configured to measure the pressure within the device at one point, at two points or more.

According to some embodiments, there is provided a pulmonary function testing device having an inlet aperture/orifice configured to provide respiratory gas from a user to the device during exhale, and from the device to the user during inhale, an outlet/exhaust aperture/orifice configured to exhaust respiratory gas from a device to the surroundings thereof (ambient air) during exhale, and to provide gas from the surrounding of the device (ambient air) to the device during inhale, and the device further has a variable/adjustable air pressure altering mechanism configured to modify (increase/decrease) the pressure of gas within the device, for example by modulating a cross-section of a conduit through which the respiratory gas flows in the device. The device further comprises a flow sensor, configured to measure the flow of gas within the device. According to some embodiments, the devices may further include a flow sensor, configured to measure the flow of gas within the device. Alternatively, according to some embodiments, the devices may further include a pressure sensor, configured to measure the pressure within the device at one point, at two points or more.

According to some embodiments, there is provided a pulmonary function testing device having an inlet aperture/orifice configured to provide respiratory gas from a user to the device during exhale, and from the device to the user during inhale, an outlet/exhaust aperture/orifice configured to exhaust respiratory gas from a device to the surroundings thereof (ambient air) during exhale, and to provide gas from the surrounding of the device (ambient air) to the device during inhale, and the device further has a compliance altering mechanism configured to the compliance of the pathways the gas passes through. The device further comprises a flow sensor, configured to measure the flow of gas within the device. According to some embodiments, the devices may further include a flow sensor, configured to measure the flow of gas within the device. Alternatively, according to some embodiments, the devices may further include a pressure sensor, configured to measure the pressure within the device at one point, at two points or more.

As used herein, the term “lung pressure” may be interchangeable with the term “alveolar pressure”, and is commonly the driving-force of the respiratory airflow.

Additional tests may be done for verifying the results and/or achieving an improved accuracy. It is noted that expirations and inspirations may be tested.

As used herein, according to some embodiments, the term “respiratory cycle” may refer to events that occur during one breath, for example, consecutive inhalation and exhalation. Other measures may be used for defining a respiration cycle, such as measured/evaluated compliance, volume, flow values or others that have a repetitive biorhythmic pattern aligned with the respiration activity, and one repetition pattern may define a cycle.

Unless specifically stated otherwise, as apparent from the following discussions, it is appreciated that throughout the specification discussions utilizing terms such as “processing”, “computing”, “calculating”, “determining”, “estimating”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices. In addition, the term “plurality” may be used throughout the specification to describe two or more components, devices, elements, parameters and the like.

Embodiments of the present disclosure may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present disclosure are not described with reference to any particular programming language. It will be appreciated that a variety of programming languages may be used to implement the teachings of the disclosure as described herein.

Although some demonstrative embodiments are described herein with relation to communication including video information, some embodiments may be implemented to perform communication of any other suitable information, for example, multimedia information, e.g., audio information, in addition to or instead of the video information. Some embodiments may include, for example, a method, device and/or system of performing wireless communication of A/V information, e.g., including audio and/or video information. Accordingly, one or more of the devices, systems and/or methods described herein with relation to video information may be adapted to perform communication of A/V information.

Some demonstrative embodiments may be implemented to communicate wireless-video signals over a wireless-video communication link, as well as Wireless-Local-Area-Network (WLAN) signals over a WLAN link. Such implementation may allow a user, for example, to play a movie, e.g., on a laptop computer, and to wirelessly transmit video signals corresponding to the movie to a video destination, e.g., a screen, while maintaining a WLAN connection, e.g., with the Internet and/or one or more other devices connected to a WLAN network. In one example, video information corresponding to the movie may be received over the WLAN network, e.g., from the Internet.

The disclosure may be described in the general context of computer-executable instructions, such as program modules, being executed by a computer. Generally, program modules include routines, programs, objects, components, data structures, and so forth, which perform particular tasks or implement particular abstract data types. The disclosure may also be practiced in distributed computing environments where tasks are performed by remote processing devices that are linked through a communications network. In a distributed computing environment, program modules may be located in both local and remote computer storage media including memory storage devices.

An exemplary system for implementing the disclosure may include a general purpose computing device in the form of a computer or computer cluster(s). Components of the computer may include, but are not limited to, a processing unit, a system memory, and a system bus that couples various system components including the system memory to the processing unit. The system bus may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Accelerated Graphics Port (AGP) bus, and Peripheral Component Interconnect (PCI) bus also known as Mezzanine bus. The computer may include a variety of computer-readable media. Computer-readable media can be any available media that can be accessed by the computer and includes both volatile and nonvolatile media, and removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by the computer. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The system memory may include computer storage media in the form of volatile and/or nonvolatile memory such as read only memory (ROM) and random access memory (RAM). A basic input/output system (BIOS), containing the basic routines that help to transfer information between elements within computer, such as during start-up, is typically stored in ROM. RAM typically contains data and/or program modules that are immediately accessible to and/or presently being operated on by a processing unit.

According to some embodiments, the terms respiration and respiratory may be used interchangeably. The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A pulmonary function testing device, the device comprising:
at least one sensor configured to provide a signal, directly or indirectly, indicative of the flow rate of air passing through said device,
a respiratory characteristic modulator configured to change between at least a first respiratory characteristic and a second respiratory characteristic; and
a processing unit configured to:
obtain from said sensor a first signal acquired during a first respiration cycle or part thereof and while the first respiratory characteristic is set;
obtain from said sensor a second signal acquired during said second respiration cycle or part thereof and while the second respiratory characteristic is set; and
derive the respiratory related parameter based at least on the first and the second signals at corresponding respiratory cycles or parts thereof.

2. The device of claim 1, wherein said at least one sensor comprises a pneumotach.

3. The device of claim 1, wherein said respiratory characteristics are selected from a group consisting of: resistance, compliance, flow and pressure.

4. (canceled)

5. (canceled)

6. The device of claim 1, wherein said respiratory characteristic modulator is an air resistance structure configured to apply a first air resistance characteristic to air passes therethrough during the first respiration cycle or part thereof and a second air resistance characteristic to air passes therethrough during the second respiration cycle or part thereof.

7. The device of claim 1, further comprising an actuator configured to control said respiratory characteristic modulator and to apply at least the first and the second respiratory characteristics.

8. The device of claim 1, wherein the respiratory related parameter comprises an airway resistance of the subject's internal airway path, lung pressure, changes in lung pressure, lung compliance, changes in lung compliance, alveolar pressure, changes in alveolar pressure or any combination thereof.

9.-14. (canceled)

15. The device of claim 1, further comprising a temperature sensor configured to measure a temperature of air inhaled or exhaled by the subject, and/or of an environment surrounding the device.

16. (canceled)

17. The device of claim 1, further comprising a temperature module configured to control a temperature of air inhaled by the subject, wherein said temperature module is configured to heat the air inhaled by the subject.

18. (canceled)

19. The device of claim 1, further comprising a humidity module configured to control humidity of air inhaled by the subject.

20. The device of claim 1, further comprising a pressure sensor configured to measure pressure of a surrounding environment of the device.

21. The device of claim 1, further comprising one or more pressure sensors configured to measure a pressure difference between two points, one of which is located in a mouthpiece or between said mouthpiece and said modulator.

22. (canceled)

23. (canceled)

24. The device of claim 1, further comprising a display unit and a user interface breath unit; wherein said user interface breath unit comprises a mask, a tube or both.

25. (canceled)

26. (canceled)

27. A pulmonary function testing system for evaluating a respiratory related parameter of a subject, the system comprising:
a respiratory characteristic modulator configured to change between at least a first respiratory characteristic and a second respiratory characteristic;
a pulmonary function testing device comprising at least one sensor configured to provide a signal, directly or indirectly, indicative of the flow rate of air passing through said device; and
a processing unit configured to:
obtain from said sensor a first signal acquired during a first respiration cycle or part thereof and while the first respiratory characteristic is set;
obtain from said sensor a second signal acquired during said second respiration cycle or part thereof and while the second respiratory characteristic is set; and
derive the respiratory related parameter based at least on the first and the second signals at corresponding respiratory cycles or parts thereof.

28.-33. (canceled)

34. The system of claim 27, wherein the respiratory related parameter comprises an airway resistance of the subject's internal airway path, lung pressure, changes in lung pressure, lung compliance, changes in lung compliance, alveolar pressure, changes in alveolar pressure or any combination thereof.

35.-40. (canceled)

41. The system of claim 27, further comprising a temperature sensor configured to measure a temperature of air inhaled or exhaled by the subject and/or of an environment surrounding the device.

42. (canceled)

43. The system of claim 27, further comprising a temperature module configured to control a temperature of air inhaled by the subject, wherein said temperature module is configured to heat the air inhaled by the subject.

44. (canceled)

45. The system of claim 27, further comprising a humidity module configured to control humidity of air inhaled by the subject.

46. The system of claim 27, further comprising a pressure sensor configured to measure pressure of a surrounding environment of the device.

47.-49. (canceled)

50. The system of claim 27, further comprising a display unit and a user interface breath unit; wherein said user interface breath unit comprises a mask, a tube or both.

51. (canceled)

52. (canceled)

53. A method for pulmonary function testing for evaluating respiratory related parameter of a subject, the method comprising:
providing a pulmonary function testing device comprising at least one sensor configured to provide a signal, directly or indirectly, indicative of the flow rate of air passing through the subject's airway;
applying a first respiratory characteristic and obtaining from the sensor a first signal, directly or indirectly, indicative of the flow rate of air passing through said device during a first respiration cycle or part thereof;
applying a second respiratory characteristic and obtaining a second signal, directly or indirectly, indicative of the flow rate of air passing through said device during said second respiration cycle or part thereof; and
deriving the respiratory related parameter based at least on the first and the second signals at corresponding respiratory cycles or parts thereof.

54.-71. (canceled)
